(19) United States
(12) Patent Application Publication
(10) Pub. No.: US 2008/0027252 A1
(43) Pub. Date: Jan. 31, 2008

(54) Oil dehalogenation method

(76) Inventors: Kermit L. Burkholder, Manheim, PA (US); Richard L. Middleton, Lancaster, PA (US)

Correspondence Address: BLANK ROME LLP, 600 NEW HAMPSHIRE AVENUE, N.W., WASHINGTON, DC, 20037, US

(21) Appl. No.: 11/493,823

(22) Filed: Jul. 27, 2006

(51) Int. Cl. C07C 5/00

(52) U.S. Cl. 585/16; 585/899

(57) ABSTRACT

Methods for dehalogenating halogenated hydrocarbons are described. The methods involve mixing the halogenated hydrocarbon with an oxidizing agent, a diol and an alkali base. The mixture is heated to a temperature sufficient to remove water from the mixture. The mixture is then reacted at a sufficient temperature for a sufficient amount of time to cause the halogens of the mixture to form halide salt solids. Solids are then removed from the mixture, leaving a dehalogenated hydrocarbon.

### FIELD OF THE INVENTION

The present invention relates to methods of dehalogenating halogenated hydrocarbons. The invention also relates to the recycling of spent oils, such as cutting oils, for other uses, such as use as heating oil.

### BACKGROUND OF THE INVENTION

Many of today's cutting oils and straight chain hydrocarbon based coolants are halogenated to prolong the life of the product. Disposal of spent halogenated hydrocarbons is problematic because of the environmental and health effects that can be caused by these hydrocarbons.

One use for spent hydrocarbons is to recycle them as industrial heating oil. Recycling spent hydrocarbons as heating oil is advantageous for at least two reasons. First, the spent hydrocarbons are given a second life as heating oil instead of being disposed of after their first use. Second, the process of burning recycled spent hydrocarbons as heating oil is a very efficient way to effect the proper disposal of the original halogenated hydrocarbon, which in some cases would be disposed of by burning at a waste treatment facility.

Most heating oil burners are not permitted by governmental regulations to burn fuel with a halogen level of greater than 1,000 parts per million (ppm). There are a very small number of burners in use that allow for the burning of oils with a halogen level of up to 4,000 ppm. Most halogenated hydrocarbons have halogen levels from 10,000 to 50,000 ppm, well above these requirements. Because of these high halogen levels, it is difficult and expensive to produce recycled heating oils that can be marketed for environmentally friendly use.

Further, the dehalogenation of hydrocarbons is one of the main barriers to the efficient disposal of contaminated hydrocarbon mixtures, as the process is often expensive and inefficient with toxic side products.

Several methods for dehalogenating spent oils and other hydrocarbons have been described in the art.

U.S. Pat. No. 5,174,893 to Halpern et al. describes a process for the dehalogenation of waste materials using a metal hydroxide and 2-methoxyethanol. The reaction is relatively efficient, dehalogenating greater than 99.9% of the original halogenated species in six hours. However, the side product of the reaction, 3,5-dichloro-1-(2-methoxy)benzene, is not environmentally friendly, and can lead to aquatic pollution.

U.S. Pat. No. 5,783,068 to Laborde et al. describes a process for dechlorination of spent lubricating oil using Group I or II metal oxides and hydroxides. The method of Laborde et al. requires the purchase of commercial chlorine trapping compounds and use of specialized distilling equipment, making the process expensive to perform.

U.S. Pat. No. 5,490,919 to Pri-Bar et al. describes a process for the dehalogenation of organohalides using an alkali hydroxide in an alcoholic solution in the presence of a catalyst and excess hydrogen. Pri-Bar et al. describe a process requiring special catalysts and reaction times of 16 hours or longer.

U.S. Pat. No. 4,776,947 to Streck et al. describes a process for the dehalogenation of a halogen containing hydrocarbon oil using an alkali or alkaline earth alcoholate. Streck et al. describe a dehalogenation process that must be performed under an inert nitrogen atmosphere, requiring specialized equipment.

The above methods have been used with varying degrees of success, convenience and cost efficiency. As such, it is desirable to develop methods that allow for safe, environmentally friendly, facile and economical dehalogenation of spent oils for their recycling.

### SUMMARY OF THE INVENTION

It is an object of the invention to provide environmentally friendly, facile and economical methods for the dehalogenation of hydrocarbons and, in particular, spent oils. The methods of the invention cause the substantial dehalogenation of the treated spent hydrocarbons. In typical use, the methods of the invention produce resultant hydrocarbons, such as heating oil, with halogen levels of 400 to 600 ppm.

The methods of the invention are economical because they use cost-effective reagents that can be stored and handled under standard laboratory conditions and do not require the use of expensive commercial catalysts or chlorine traps. They are facile because they can be performed using standard laboratory equipment.

The methods of the invention are also environmentally friendly because non-toxic halogen salt side products are produced along with dehalogenated hydrocarbons. These halogen salts can be easily and safely disposed of or can be further applied to other commercial uses.

It is a further object of the invention to provide a method for dehalogenation of spent hydrocarbons through use of oxidizing agents. Methods of the invention utilize oxidizing agents, such as permanganate salts to safely and easily dehalogenate hydrocarbons of various types.

The foregoing objects and advantages are accomplished according to the invention by a method wherein the halogenated hydrocarbon is mixed with an oxidizing agent, a diol and an alkali base and the mixture is heated to a temperature sufficient to cause dehalogenation of the halogenated hydrocarbon.

### DETAILED DESCRIPTION OF THE INVENTION

Various halogenated hydrocarbons can be dehalogenated by the methods of the invention. Non-limiting examples of hydrocarbons that can be dehalogenated are cutting oils, lubricating oils, and heating oils, polychlorinated biphenyls (PCBs), chlorofluorocarbons, chlorinated paraffins, hydrochlorofluorocarbons, hydrofluorocarbons, perfluorocarbons and halons. The methods of the invention may also be used to dehalogenate halogenated hydrocarbons for their further use or for disposal. Typical starting materials for the methods of the invention will have halogen levels of 10,000 to 40,000 ppm. Use of starting materials with lower or higher halogen levels is also contemplated.

In a preferred embodiment, halogenated oils are treated by the methods of the invention to form heating oils which can then be used in conventional heating oil burners. The heating oils that are produced by the invention will preferably have halogen levels no greater than about 600 ppm. However, it is also contemplated that the heating oils produced by the invention might have higher halogen levels, such as less than about 1000 ppm or less than about 4000 ppm. It is most important that the resultant heating oils have halogen levels that meet the governmental and local standards for heating oils for use in the burner in which they will be consumed.

The methods of the invention cause dehalogenation of halogenated hydrocarbons through an oxidative process in which the halogenated hydrocarbons are mixed with oxidizing agents. Preferred oxidizing agents are permanganate salts, most preferably potassium permanganate. Use of other oxidizing agents is also contemplated, including, but not limited to peroxides.

The methods of the invention involve mixing the halogenated hydrocarbon solution with a diol. Preferably, the diol used in the invention is propylene glycol(1,2-propane diol). However, the use of other diols, such as ethylene glycol or polyethylene glycol, is also contemplated.

The methods of the invention also involve mixing the halogenated hydrocarbon solution with an alkali base. Preferably, the alkali base used in the invention is potassium hydroxide. However, the use of other alkali bases, such as sodium hydroxide, is also contemplated.

In a preferred method of the invention, the halogenated hydrocarbons are dehalogenated by reacting the halogenated hydrocarbon solution with sufficient amounts of potassium permanganate, propylene glycol and potassium hydroxide to cause dehalogenation of the hydrocarbons. The amount of each reagent added to the reaction is dependent on the halogen level of the hydrocarbon solution being treated, i.e.—more of each reagent will need to be added to dehalogenate hydrocarbons with higher halogen levels.

The halogen level of the hydrocarbon solution can be determined by a number of methods well known in the art. The halogen levels of hydrocarbon samples may be analyzed by gas chromatography methods or mass spectrometry methods. For example, the halogen level of the hydrocarbon solution may be analyzed using Method 9076 of publication number SW-845 from the United States Environmental Protection Agency (available at http://www.epa.gov/epaoswer/hazwaste/test/main.htm).

According to a preferred embodiment, after the halogen level is determined, reagents may be added according to the following example ratios:

about 0.60 g to about 1.0 g of potassium permanganate for every 1 g of halogen in the sample;

about 6.0 g to about 10.0 g of propylene glycol for every 1 g of halogen in the sample; and

about 2.0 g to about 7.0 g of potassium hydroxide for every 1 g of halogen in the sample.

It is also contemplated that the ratios of reagent to halogen in the sample may vary. They may be lower or higher as is necessary to reduce the halogen level of the sample to the final desired level. As a general rule, higher ratios of reagent to halogen in the sample will lead to lower halogen levels in the oil produced.

Initially, the reagents and the halogenated hydrocarbon are mixed in a mixing vessel. The mixing vessel may be any vessel suitable for mixing and heating of the halogenated hydrocarbon and the reagents to be used. The mixing vessel may be made of glass, metal or other suitable material.

After the mixture is formed, it is heated from ambient temperature to an elevated temperature sufficient to remove water, for example, about 250° F. Once the temperature of the mixture reaches 250° F., water should be substantially removed from the mixture. Alternatively, the mixture could be heated to a lower temperature, such as 225-230° F. and allowed to stand at that temperature until substantially all water is removed from the mixture. For example, the mixture could be heated at 230° F. for 1 hour to substantially remove all water from the mixture.

After heating, the mixture is then moved to a reaction vessel. In a preferred embodiment, the reaction vessel is a reactor with a carbon steel, jacketed tank with a mixer and an expansion tank. Preferably, the expansion tank used is of equal volume to the volume of the reactor. It is also preferred that the volume of the mixture added to the reactor be approximately one third of the volume of the reactor. It is also contemplated that the reaction vessel may also be another type of reactor, or any type of vessel that can safely contain and heat the reactants.

In the reactor, the mixture is slowly heated to an elevated temperature sufficiently high to promote the reaction, preferably 450° F., at atmospheric pressure. The temperature of the mixture is maintained for a period of time sufficient to allow the reaction to occur. Preferably, the mixture is maintained at about 450° F. for a period of about three hours. The reaction temperature may be varied, with the caveat that the minimum temperature for promoting the reaction is about 350° F. At different temperatures, the reaction will require different reaction times. For example, at 350° F., the reaction should take about eight hours; at 425° F., it should take about five hours; and at 500° F. it should take about two hours.

During the reaction, the halogens of the mixture combine with potassium (or other alkali metal) to form halide salts. The halide salts that are formed are harmless and can be easily disposed of after their removal from the mixture, as described below.

After the reaction, the halide salts must be removed from the dehalogenated oil. It is also likely that the oil will be contaminated with other solids from its original use. Such solids might include chips and shavings of metal, swarf and other materials. It is preferred that the other solids be removed at the same time as the removal of halide salts. Preferably, the solids are removed using a settling tank, as described below. If all of the solids are removed at the same time, an extra step in the dehalogenating process is avoided, reducing costs and material handling requirements.

The methods of the present invention use reagents that allow for efficient and safe dehalogenation even in the presence of solids in the original hydrocarbon sample. However, these other solids may be removed before the dehalogenation process of the invention. Removal of solids before dehalogenation may be desireable if the solids contained in the hydrocarbon sample might interfere with the dehalogenating process, or if they might react dangerously with the reagents of the invention. If necessary, the solids may be removed through any common method known in the art for removing particles from oils, such as the methods mentioned below.

In a preferred embodiment, the dehalogenated hydrocarbon solution is transferred from the reaction vessel to a settling tank, such as a conical bottom settling tank. The solids in the mixture are allowed to settle, leaving high quality dehalogenated oil. Alternatively, the dehalogenated hydrocarbon sample may be transferred from the settling tank to a decanting centrifuge for the removal of halide salts and other solids.

Specific examples of the method of the invention are set forth below. These examples are meant to further illustrate the invention and are not intended to limit the scope and spirit of the invention as presented in the claims. Although the examples given are for the dehalogenation of spent oils, it should be apparent that the method of the invention can also be used for dehalogenating other halogenated hydrocarbons.

### EXAMPLES

### Example 1

Used raw cutting oil with a halogen level of 13,900 mg/L was mixed in a mixing vessel with reagents as follows:

| | |
|---|---|
| Oil sample size | 1,000 mL |
| 6.5% Potassium Permanganate | 150 mL |
| Propylene glycol | 100 mL |
| 50% Potassium Hydroxide | 100 mL |

The reaction mixture was pre-heated to 250° F. to remove water. The mixture was then moved to a 3.0 L carbon steel jacketed reactor with a mixer and expansion tank. The mixture was reacted with agitation at 450° F. for three hours. After completion of the reaction, the mixture was transferred to a conical bottom settling tank where solids in the mixture were allowed to settle out. The resultant oil was removed from the settling tank, leaving the solids. The final product oil had a halogen level of 376 mg/L.

### Example 2

Used raw cutting oil with a halogen level of 8,400 mg/L was mixed in a mixing vessel with reagents as follows:

| | | |
|---|---|---|
| Oil sample size | 1,000 | mL |
| 6.5% Potassium Permanganate | 100 | mL |
| Propylene glycol | 50 | mL |
| 50% Potassium Hydroxide | 50 | mL |

The mixture was reacted as in Example 1. The final product oil had a halogen level of 375 mg/L.

### Example 3

Used raw cutting oil with a halogen level of 4,600 mg/L was mixed with reagents as follows:

| | | |
|---|---|---|
| Oil sample size | 1,000 | mL |
| 6.5% Potassium Permanganate | 50 | mL |
| Propylene glycol | 25 | mL |
| 50% Potassium Hydroxide | 25 | mL |

The mixture was reacted as in Example 1. The final product oil had a halogen level of 579 mg/L.

What is claimed is:

1. A method for dehalogenating a halogenated hydrocarbon comprising:
forming a mixture comprising the halogenated hydrocarbon, an oxidizing agent, a diol, and an alkali base; and
heating the mixture to a temperature sufficient to cause dehalogenation of said halogenated hydrocarbon.

2. The method for dehalogenating a halogenated hydrocarbon of claim 1, wherein the oxidizing agent is potassium permanganate.

3. The method for dehalogenating a halogenated hydrocarbon of claim 1, wherein the diol is propylene glycol.

4. The method for dehalogenating a halogenated hydrocarbon of claim 1, wherein the alkali base is potassium hydroxide.

6. The method for dehalogenating a halogenated hydrocarbon of claim 1, further comprising removing solids from the resultant dehalogenated hydrocarbon solution.

7. The method for dehalogenating a halogenated hydrocarbon of claim 1, wherein the resultant dehalogenated hydrocarbon has a halogen level of less than about 600 ppm.

8. The method for dehalogenating a halogenated hydrocarbon of claim 1, wherein the resultant dehalogenated hydrocarbon has a halogen level of less than about 1000 ppm.

9. The method for dehalogenating a halogenated hydrocarbon of claim 1, wherein the resultant dehalogenated hydrocarbon has a halogen level of less than about 4000 ppm.

10. The method for dehalogenating a halogenated hydrocarbon of claim 1, wherein, prior to heating the mixture to a temperature sufficient to cause dehalogenation, the mixture is heated to a temperature sufficient to remove water from the mixture.

11. A method for dehalogenating a halogenated hydrocarbon comprising:
forming a mixture comprising the halogenated hydrocarbon, potassium permanganate, propylene glycol, and potassium hydroxide;
heating the mixture to a temperature sufficient to eliminate water from the mixture; and
reacting the mixture at a temperature sufficient to cause dehalogenation of the halogenated hydrocarbon.

12. The method for dehalogenating a halogenated hydrocarbon of claim 11, wherein the temperature sufficient to eliminate water from the reaction mixture is about 250° F.

13. The method for dehalogenating a halogenated hydrocarbon of claim 11, wherein the minimum temperature sufficient to cause dehalogenation is about 350° F.

14. The method for dehalogenating a halogenated hydrocarbon of claim 11, wherein potassium permanganate is added to the mixture in a ratio of about 0.60 g to about 1.0 g of potassium permanganate for every 1 g of halogen in the hydrocarbon.

15. The method for dehalogenating a halogenated hydrocarbon of claim 11, wherein propylene glycol is added to the mixture in a ratio of about 6.0 g to about 10.0 g of propylene glycol for every 1 g of halogen in the hydrocarbon.

16. The method for dehalogenating a halogenated hydrocarbon of claim 11, wherein potassium hydroxide is added to the mixture in a ratio of about 2.0 g to about 7.0 g of potassium hydroxide for every 1 g of halogen in the hydrocarbon.

17. The method for dehalogenating a halogenated hydrocarbon of claim 11, wherein the resultant dehalogenated hydrocarbon solution has a halogen level of less than 600 ppm.

18. The method for dehalogenating a halogenated hydrocarbon of claim 11, wherein the resultant dehalogenated hydrocarbon solution has a halogen level of less than 1000 ppm.

19. The method for dehalogenating a halogenated hydrocarbon of claim 11, wherein the resultant dehalogenated hydrocarbon solution has a halogen level of less than 4000 ppm.

20. A method for dehalogenating a halogenated hydrocarbon sample comprising
forming a mixture comprising the halogenated hydrocarbon sample and an oxidizing agent; and
heating the mixture to a temperature sufficient to cause dehalogenation of said halogenated hydrocarbon.

21. A dehalogenated hydrocarbon product formed from the method of claim 1.

22. The product of claim 21, wherein said dehalogenated hydrocarbon product is a heating oil.

23. The product of claim 21, wherein said dehalogenated hydrocarbon product is a lubricating oil.

24. The product of claim 21, wherein said dehalogenated hydrocarbon product is a cutting oil.
